(19) United States
(12) Patent Application Publication
(10) Pub. No.: US 2007/0265506 A1
(43) Pub. Date: Nov. 15, 2007

(54) TWO-TIER MODEL TO SCREEN PATIENTS WITH SLEEP-DISORDERED BREATHING

(76) Inventors: Pa-Chun WANG, Taipei (TW); Ning-Hung Chen, Taipei (TW)

Correspondence Address: LEONG C LEI, PMB # 1008, 1867 YGNACIO VALLEY ROAD, WALNUT CREEK, CA, 94598, US

(21) Appl. No.: 11/382,483

(22) Filed: May 10, 2006

(51) Int. Cl. G06F 19/00; A61B 5/00

(52) U.S. Cl. 600/300; 705/3

(57) ABSTRACT

A two tier model for screening patients with sleep-disordered breathing includes collecting clinical information of a number of patients are collected, including gender, age, and body mass index, and performing form surveys including the Epworth Sleepiness Scale and the Snore Outcomes Survey to obtain a respiratory disturbance index (RDI). Receiver operating characteristics (ROC) are calculated with an initial strategy to maximize prediction sensitivity for patients with obstructive sleep apnea syndrome(OSAS). The associations between pulse oximeter data (desaturation index of 3%, DI3) against RDI was the second strategy to maximize prediction specificity.

### BACKGROUND OF THE INVENTION

(a) Technical Field of the Invention

The present invention relates to a method for screening patents with sleep-disordered breathing (SDB), and in particular to a two-tier prediction method for screening of sleep-disordered breathing adults.

(b) Description of the Prior Art

Sleep disordered breathing (SDB) is a disease in prevalence among middle-aged population. SDB patients are at higher risk to develop cardiovascular consequence and neuro-cognitive dysfunction. SDB can also raise the risks of traffic and working place accidents. Increasing awareness of the adverse outcomes associated with SDB has led to a rapid rise in the demand of diagnostic polysomnography (PSG).

Owing to the insufficient capacity and long waiting time for PSG, several attempts have been made to develop screening approaches with an intention to simplify diagnostic procedures and to reduce costs by the use of home-based screening tools. Studies based on single individual indices such as clinical features, questionnaires, or pulse oximetry have been conducted to predict SDB with successes to some extent. Unfortunately, there has been little consensus in regard to the most reliable set of clinical features that can differentiate the absence or presence of SDB. The association algorithms have been formulated using self-reported SDB symptoms with high sensitivity but low specificity; carrying the handicap in reducing actual PSG numbers. Pulse oximetry, however, is less sensitive but highly specific.

A simple but effective screening system can help clinicians to prioritize patients for full over-night PSG. It is believed that a stepwise approach with proper risk stratification strategy can overcome the limitation of individual screening tools to optimize effectiveness of the whole prediction algorithm. Hence, the present invention is aimed to develop a two-tier screening model for adult patients with SDB.

### SUMMARY OF THE INVENTION

The primary purpose of the present invention is to provide a two-tier screening method for adult patients with SDB, wherein in the first tier screening, a basic clinical information (gender, age, and body mass index-BMI), Epworth Sleepiness Scale (ESS), and Snore Outcome Survey (SOS) is formulated with an aim to maximize screening sensitivity, and patients with low risk for sleep apnea will be exempted from PSG testing. In the second tier screening, pulse oximeter is employed to identify patients with high risk for severe sleep apnea by maximizing screening specificity. The two-tier screening strategy is used to exclude patients at low risks of sleep apnea, and to prioritize patients at high risks of severe sleep apnea for early PSG testing.

Another objective of the present invention is to provide a two-tier screening method for adults with SDB, which, besides effectively screening out SDB patients, is also suitable for large-scale community and occupational screening purposes.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

The present invention will be apparent to those skilled in the art by reading the following description, with reference to the attached drawings, in which:

FIG. 1 shows a receiver operating characteristic curve using gender, age, BMI, SOS, and ESS against OSAS (RDI≧5). (area under curve 0.88, standard error 0.026, Z 14.62, p<0.001);

FIG. 2 shows RDI vs. estimated probability of having OSAS (RDI≧5) when all independent predictors are incorporated in the logistic regression, wherein 86.20% of patients whose predicted probability of having OSAS is higher than 60%;

FIG. 3 shows a receiver operating characteristic curve using DI3 (desaturation index of 3%) against severe OSAS (RDI≧30). (area under curve 0.951, standard error=0.024, Z=18.792, p<0.001), wherein for DI2 and DI4 (desatuartion index 2 and 4%), the AUC are similar (0.942, with standard error=0.027, Z=16.3763, p<0.001);

FIG. 4 is a simple linear regression model shows that DI3 (β=1.207, p<0.001, adjusted R²=0.833) and RDI are strongly correlated; and

FIG. 5 is a plot of probability of having severe OSAS (RDI≧30) when DI3 is introduced into the logistic regression analysis, among those whose predicted probability greater than 0.5, 96% being truly severe OSAS patients and 4% being misclassified.

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

In accordance with the present invention, a number of patients (aged 18-80 years), for example 355 patients, are evaluated in a consecutive manner to determine the presence of SDB. The patients' demographic and characteristics data are collected upon entry. The patients are all administered with snore outcomes survey (SOS) and Epworth sleepiness scale (ESS).

The patients all receive standard overnight in-lab polysomnography (Nicolet, Nicolet Inc. Madison, Wis.) to obtain at least six (6) hours of sleep data recording. The respiratory disturbance index (RDI) obtained from polysomnography is used as golden standard for data analysis. RDI is defined as the sum of total apnea and hypopnea episodes per hour of sleep. An apnea episode is defined as cessation of airflow lasting longer than 10 seconds, whereas a hypopnea episode is defined as a 50% or greater reduction in combined oral and nasal flow lasting longer than 10 seconds. A RDI of 5 episodes/hour is used as the cut-off point; patients with a RDI of 5 episodes/hour or less are considered as simple snorer (with no sleep apnea) and the apnea group would constitute patients with RDI greater than 5 episodes/hour. Patients with RDI of over 30 episodes/hour are considered of having severe sleep apnea.

The snore outcomes survey (SOS) is a validated outcome measure to evaluate the health impact and treatment effectiveness for adults with SDB and snoring. The SOS contains eight (8) items that evaluate the duration, severity, frequency, and consequences of problems associated with SDB on a Likert scale, each item having 5 to 6 response options. The SOS total score is transformed into a scale ranging from 0 (worst) to 100 (best).

An 8-item Epworth sleepiness scale (ESS) is used for evaluating adults on the average sleep propensity in daily life. Scores for each of the 8 items can range from 0 to 3 and the total Epworth score ranges from 0 to 24 (lowest to highest sleep propensity). The reliability, unitary structure and validity of the ESS are supported by experimental evidences in distinguishing the excessive daytime sleepiness of narcoleptics from that of normal subjects.

First Tier Screening Modeling

A multiple regression is applied to investigate the association between RDI and various OSAS-related factors. Specifically, RDI is modeled as a function of gender, age, BMI, SOS and ESS.

While RDI is dichotomized as “RDI” for RDI<=5 vs. “non-RDI” for RDI>5, a multiple logistic regression is used to examine the possibility of having greater RDI and OSAS-related factors after adjusting for gender, age and BMI.

Receiver operating characteristic (ROC) curve is used to determine the diagnostic thresholds for SOS and ESS that are more likely to differentiate “OSAS” From “non-OSAS”. The area under curve (AUC) is demonstrated. The sensitivity, specificity, positive and negative predictive values (PPV and NPV) of different possible SOS and ESS combinations is calculated. The boot-trap technique is used to identify the cut-off point, the optimal SOS and ESS combination in order maximize the sensitivity of the model to include as many OSAS patients as possible.

A pulse oximeter, such as Pulsox-3i (Minolta Co., Ltd., Osaka, Japan) is used for home oxygen saturation monitoring. This pulse oximeter is a portable device designed to measure SpO₂ (saturated arterial oxygen pressure), pulse rate, and pulse strength during sleep that has 12-hour data memory function. Desaturation of oxygen by 2, 3, and 4% (oxygen desaturation index of 2, 3, and 4%; DI2, DI3, and DI4) is defined as an episode of respiratory disturbance in the method in accordance with the present invention. All the patients received pulse oximeter examination simultaneously with in-lab polysomnography. The receiver operating characteristic (ROC) curve is then used to determine the most accurate diagnostic desaturation thresholds to differentiate “severe OSAS” from “non=severe OSAS”.

Second Tier Screening Modeling

One hundred (100) possible OSA patients that have been identified of having OSAS (predicted positive for RDI≧5) in the first tier screening are randomly selected for pulse oximeter examination. The patients undergo overnight (at least 6 hours) Pulsox-3i monitoring and recording. The sleep oxygen desaturation events data were retrieved and stored using Pulsox-3 DS-3 Data Analysis (Minolta Co., Osaka, japan) software.

Similar to the regression model in the first tier screening, the multiple and logistic regression are used to evaluate the relationship between RDI and DI3 for continuous and binary RDI, respectively. It is noted that binary RDI in the second screening is defined as “severe OSAS ”with RDI>=30 vs. “non-severe OSAS” with RDI<30.

The receiver operating characteristic (ROC) curve is used to determine the most appropriate diagnostic threshold of DI3 that can differentiate “severe OSAS” from “non-severe OSAS”. The area under curve (AUC) is demonstrated. The sensitivity, specificity, PPV and NPV of DI3 are also tabulated. The optimal DI3 cut-off point would maximize the specificity of the second tier screening model, without sacrificing its sensitivity, to exclude as many “non-severe OSAS” patients as possible.

All data are stored in Access 7.0 database (Microsoft, Redmond, Seattle) and are analyzed using the SAS software package (SAS Institute, Cary, N.C.). A p value of<0.05 was considered to be statistically significant. A multiple regression is used to model a continuous variable on all possible covariates. For dichotomous variable of interest, a multiple logistic regression is then employed to address the association between variables.

Result

In the study of the present invention, the initial study group consists of 355 patients, of which 312 (87.9%) are male and 43 (12.1%) are female. The mean RDI is 38.3±29.9 episodes/hr, and 48 (13.5%) patients do not have OSAS (RDI<5 episodes/Hr), while as 69 (19.4%) have RDI≧5 but <15 episodes/hr, 52 (14.6%) have RDI≧15 but <30 episodes/hr, and 186 (52.4%) have RDI≧30. Patients' age, gender, body mass index (BMI), SOS, and ESS scores are all significantly correlated with RDI (Table 1).

TABLE 1
Patients' Demographics and Survey Score

| Variable | Mean ± SD | γ (p value*) | |
|---|---|---|---|
| Age (years-old) | 44.7 ± 11.3 | 0.101 | (.056) |
| BMI (kg/m²) | 27.4 ± 4.1 | 0.405 | (<.001) |
| SOS | 44.9 ± 15.3 | −0.412 | (<.001) |
| ESS | 10.9 ± 5.2 | 0.253 | (<.001) |

*Pearson's correlation coefficient.
Note:
The mean RDI is 23.31 ± 32.19 episodes/hr of female and 40.21 ± 29.28 episodes/hr of male, the p value of t-statistic from 2-sample t-test is .000. ESS: Epiworth Sleepiness Scale, SOS Snore Outcomes Survey

First Tier Screening Prediction

The multiple regression reveals that gender, age, BMI, SOS, and ESS are all significant predictors of RDI and the adjusted R₂ for this model is 0.286 (Table 2).

TABLE 2
Predictors for RDI (Multiple Regression Analysis)

| | Estimated β | p value |
|---|---|---|
| Gender (male) | 8.179 | 0.054 |
| Age | 0.269 | 0.024 |
| BMI | 2.228 | <0.001 |
| ESS | 0.538 | 0.051 |
| SOS | −.573 | <0.001 |

The estimated RDI is:
est RDI=−13.914+8.179Xₛₑₓ+0.269X_{age}+2.228X_{BMI}+0.538X_{ESS}−0.573X_{SOS}.

where sex=1 and 0 for male and female, respectively.

The significant factors in previous model are also predictors of the probability of having OSAS (RDI≧5) (Table 3).

TABLE 3
Predictors for Having OSAS (Logistic Regression)

| | Estimated | | 95% Conf. | |
|---|---|---|---|---|
| | β | Odds Ratio | Interval | p value |
| Gender | 1.096 | 2.99 | 1.05-8.55 | 0.041 |
| (male) | | | | |
| Age | 0.064 | 1.07 | 1.03-1.11 | 0.001 |
| BMI | 0.264 | 1.30 | 1.15-1.47 | <0.000 |
| ESS | 0.039 | 1.04 | 0.96-1.13 | 0.34 |
| SOS | −0.062 | 0.94 | 0.92-0.97 | <0.000 |

Note that “gender” and “ESS” are less significant in predicting continuous RDI than in predicting binary RDI. However, the significance is very close. Based on this model, the probability of having OSAS is:

$$\hat{P} ( having OSAS ) = \frac{{ⅇ}^{- 5.935 + 1.096 {X}_{sex} + 0.064 {X}_{age} + 0.264 {X}_{BMI} + 0.039 {X}_{ESS} - 0.062 {X}_{SOS}}}{1 + {ⅇ}^{- 5.935 + 1.096 {X}_{sex} + 0.064 {X}_{age} + 0.264 {X}_{BMI} + 0.039 {X}_{ESS} - 0.062 {X}_{SOS}}}$$

$$p = {ⅇ}^{k} / ( 1 + {ⅇ}^{k} )$$

$$k = - 5.935 + 1.096 {X}_{sex} + {0.064}_{age} + {0.264}_{BMI} + 0.039 {X}_{ESS} - 0.062 {X}_{SOS}$$

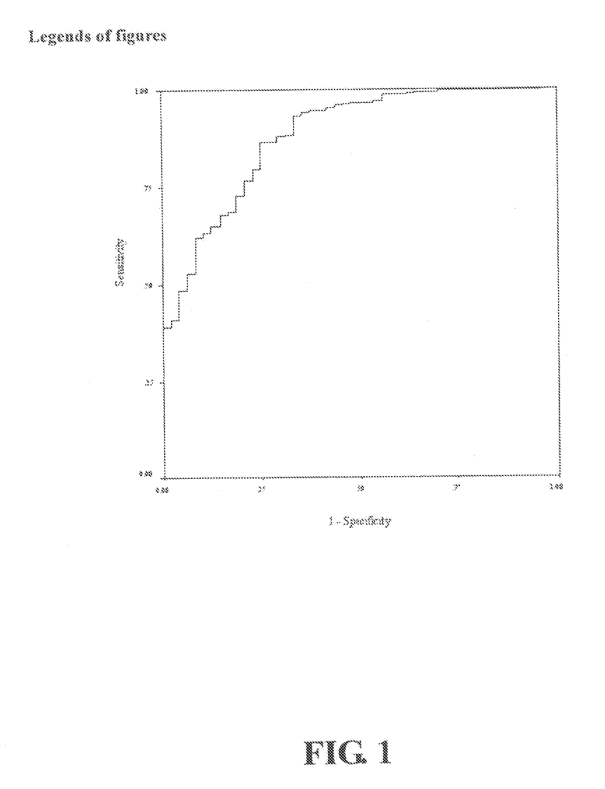

FIG. 1 shows the ROC curve of the first tier screening model. The sensitivity, specificity, PPV, and NPV of different possible SOS/ESS combinations in predicting OSAS are shown in Table 4.

TABLE 4
Relative Discriminatory Powers of ESS and SOS

| Surveys' Scores | Sensitivity | Specificity | PPV % | NPV % |
|---|---|---|---|---|
| ESS ≧ 9, SOS ≦ 40 | 0.381 | 0.833 | 93.60% | 17.39% |
| ESS ≧ 9, SOS ≦ 45 | 0.495 | 0.792 | 93.83% | 19.69% |
| ESS ≧ 9, SOS ≦ 50 | 0.541 | 0.75 | 93.26% | 20.34% |
| ESS ≧ 9, SOS ≦ 55 | 0.603 | 0.729 | 93.43% | 22.29% |
| ESS ≧ 10, SOS ≦ 40 | 0.358 | 0.917 | 96.49% | 18.26% |
| ESS ≧ 10, SOS ≦ 45 | 0.453 | 0.875 | 95.86% | 20.00% |
| ESS ≧ 10, SOS ≦ 50 | 0.498 | 0.833 | 95.00% | 20.51% |
| ESS ≧ 10, SOS ≦ 55 | 0.538 | 0.813 | 94.83% | 21.55% |
| ESS ≧ 11, SOS ≦ 40 | 0.326 | 0.917 | 96.15% | 17.53% |
| ESS ≧ 11, SOS ≦ 45 | 0.407 | 0.896 | 96.15% | 19.11% |
| ESS ≧ 11, SOS ≦ 50 | 0.437 | 0.854 | 95.04% | 19.16% |
| ESS ≧ 11, SOS ≦ 55 | 0.472 | 0.833 | 94.77% | 19.80% |
| ESS ≧ 12, SOS ≦ 40 | 0.296 | 0.958 | 97.85% | 17.56% |
| ESS ≧ 12, SOS ≦ 45 | 0.375 | 0.938 | 97.46% | 18.99% |
| ESS ≧ 12, SOS ≦ 50 | 0.401 | 0.917 | 96.85% | 19.30% |
| ESS ≧ 12, SOS ≦ 55 | 0.437 | 0.896 | 96.40% | 19.91% |

It is found that the combination of “SOS=55 and ESS=9” is an optimal cut-off point that yields relatively higher sensitivity (0.603) and specificity in this first-tire screening model.

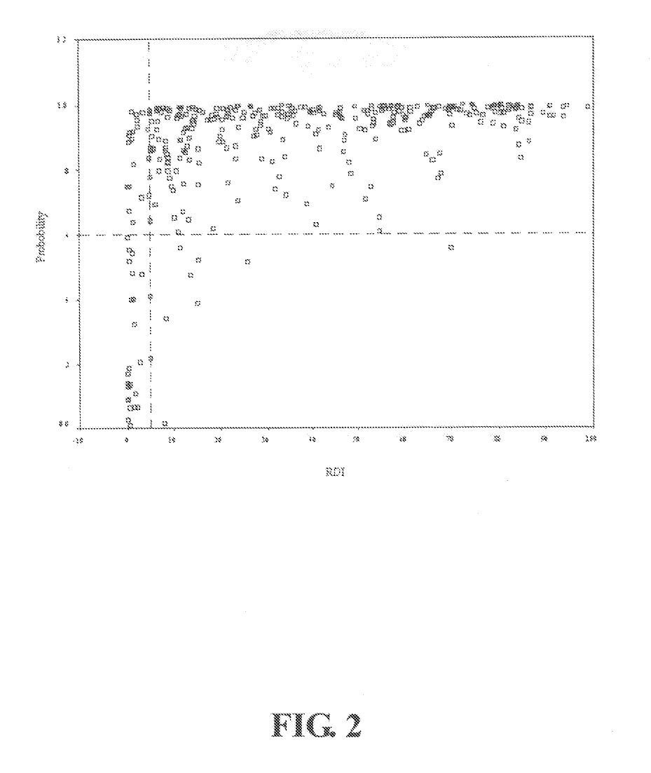

A calculated probability of 0.6 (see FIG. 2) would increase as many patients (n=337, 94.93%) as possible that have a PPV of 0.997 (306/307) for the diagnosis of OSAS (Table 5).

TABLE 5
First-Tier Screening Model Predictability

| | Predicted Positive | Predicted Negative |
|---|---|---|
| True Positive (n = 307) | hit 306 | miss 1 |
| True Negative (n = 48) | false alarm 31 | hit 17 |

Second Tier Screening Prediction

The second tier screening study group consists of 100 patients that are randomly selected from the predicted positive population (RDI≧5, presumably having OSAS, n=337) of the first tier screening. There are 83 (83%) male and 17 (17%) female. The mean age is 43.3±11.5 years-old and the BMI is 26.5±3.7. The mean RDI is 32.2±28.4 episodes/hr and 19 (19%) patients do not have OSAS (RDI<5 episodes/Hr), while as 21(21%) have RDI≧5 but <15 episodes/hr, 18 (18%) have RDI≧15 but <30 episodes/hr, and 42 (42%) have RDI≧30. The mean DI3 of this cohort is 22.3±21.5%.

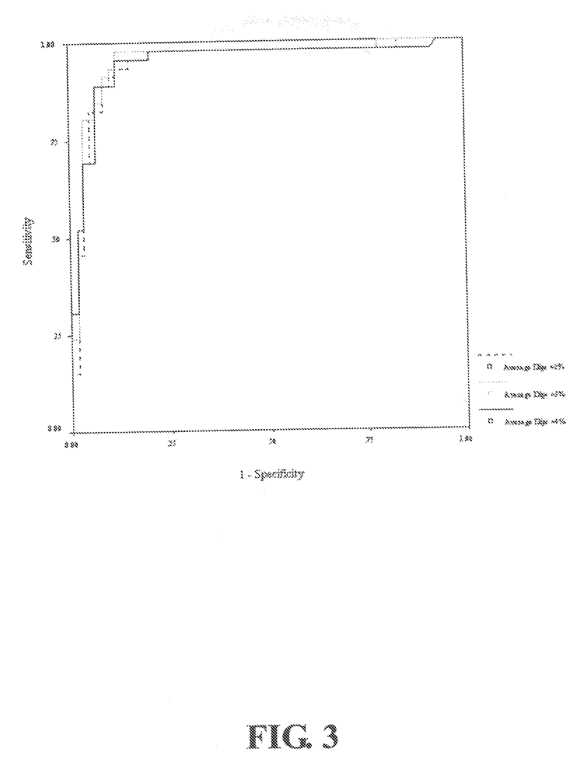

The ROC curve using DI3 against severe OSAS (RDI≧30) shows that the area under curve (AUC is 0.951 (standard error=0.024, Z=18.792, p<0.001). The ROC curves using DI2 and DI4 against severe OSAS (RDI≧30) show that the area under curve AUC is 0.942 (standard error=0.027, Z=16.3763, p<0.001) for DI2, and similarly, the area AUC is 0.942 (standard error=0.027, Z=16.3763, p<0.001) for DI4. The DI3 is therefore chosen for desaturation index in this study (FIG. 3).

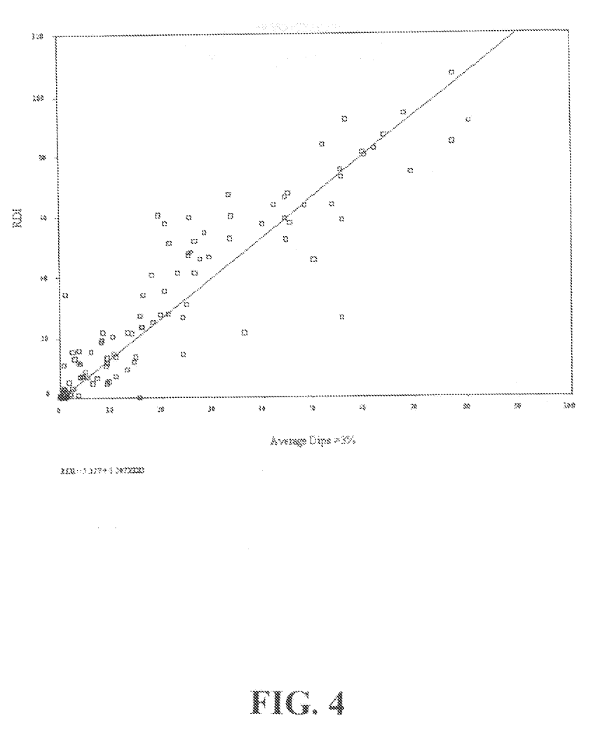

The linear regression analysis shows that DI3 is positively associated with RDI (p<0.001) and the adjusted R² for this model is as high as 0.833 (FIG. 4). As we expect, DI3 dominates the variation of RDI over other variables that are significant in the first-tier screening like gender, age, BMI and SOS.

The estimated RDI is: est RDI=5.327+1.207X_{DI3} The logistic regression model shows that DI3 is positively related to the possibility of having severe OSAS (RDI≧30) (estimated beta=0.170, p<0.001), and the probability of having server OSAS is:

$$\hat{P} ( having severe OSAS ) = \frac{{ⅇ}^{- 3.627 + 0.170 {X}_{DI 3}}}{1 + {ⅇ}^{- 3.627 + 0.170 {X}_{DI 3}}}$$

$$p = {ⅇ}^{k} / ( 1 + {ⅇ}^{k} )$$

$$k = - 3.627 + 0.170 {X}_{DI 3}$$

The ROC curve using DI3 against severe OSAS (RDI≧30) shows that the area under curve (AUC) is 0.951 (standard error=0.024, Z=18.792, p<0.001) (FIG. 4). The sensitivity, specificity, PPV, and NPV of DI3 in predicting severe OSAS are shown in Table 6.

TABLE 6
Relative Discriminatory Powers of DI3
for Severe OSAS (RDI ≧ 30)

| DI3 | | | | |
|---|---|---|---|---|
| (episodes/hr) | Sensitivity | Specificity | PPV % | NPV % |
| 5 | 0.976 | 0.448 | 75.93% | 97.83% |
| 10 | 0.976 | 0.655 | 78.43% | 95.92% |
| 20 | 0.905 | 0.914 | 81.63% | 96.08% |
| 30 | 0.571 | 0.966 | 82.98% | 94.34% |
| 40 | 0.357 | 0.983 | 84.78% | 94.44% |
| 50 | 0.075 | 0.994 | 86.67% | 94.55% |

It is found that DI3=30 would optimize specificity (0.966) of this second tire screening model to exclude as many non-severe OSAS patients as possible.

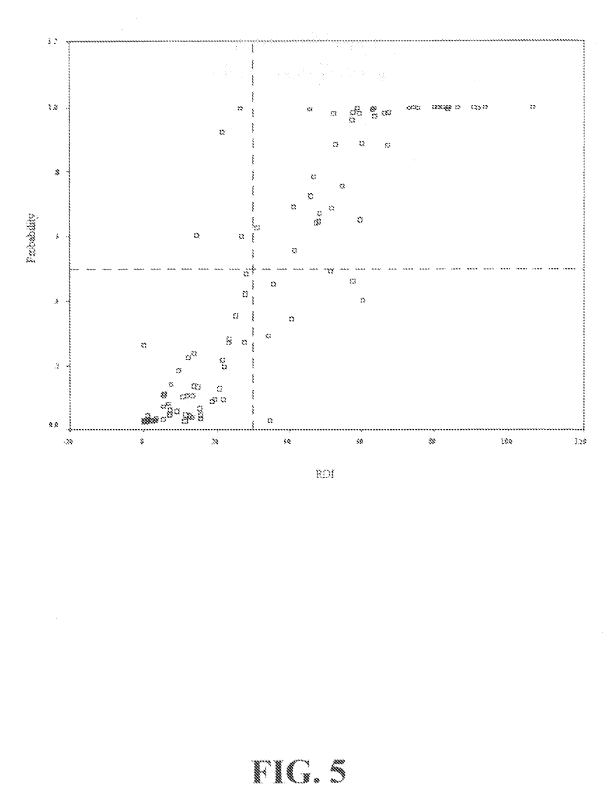

With a NPV of 0.93(54/58) (Table 7) and a calculated probability of 0.5 (FIG. 5), this second tier screening model would exclude as many patients (n=54, 54%) as possible that do not have severe OSAS.

TABLE 7
Second Tier Screening Model Predictability

| | Predicted Positive | Predicted Negative |
|---|---|---|
| True Positive (n = 42) | to 36 | miss 6 |
| True Negative (n = 58) | false alarm 4 | hit 54 |

Patients with snoring or apnea often show increased difficulties with concentration, learning new tasks, and performing monotonous tasks. Disturbed sleep at night can lead to problems with daytime attention and work performance. Lindberg et al. found that men who reported both snoring and excessive daytime sleepiness are at an increased risk of occupational accidents (odds ratio 2.2). Ulfberg J et al. concluded that the risk of being involved in an occupational accident was about 2fold among male, 3fold among female heavy snorers and increased by 50% among those suffering from OSAS. SDB is also linked to increased traffic accidents. Powell et al. estimated that sleep disorders were reported by 22.5% of all respondents who had involved with motor vehicle accident. Young T et al. found men with AHI>5 were significantly more likely to have at least one accident in 5 years (adjusted odds ratio=3.4 for habitual snorers, 4.2 for AHI 5-15, and 3.4 for AHI>15). Men and women combined with AHI>15 were significantly more likely to have multiple accident in 5 years (odds ratio=7.3). Hence, in order to reduce professional liability, it is of utmost importance for the government or cooperate authorities to early identify patients at highest risks of severe SDB.

In combination with clinical information (such as age, gender, BMI, or cephalometric data), standard sleep questionnaires or clinical index scores have been tried to describe the prevalence of snoring, observed apneas, and daytime sleepiness in general population; and to describe the relationships of these sleep disturbances to health status. For example, West et al. used BMI and ESS to prioritize patients for PSG study, they claimed to have successfully reduced the average waiting times to sleep study by approximately 90 days and to nasal CPAP trial by 32 days. In the present invention, the widely circulated ESS and SOS, which cover two important but distinct dimensions (sleepiness and snoring) of SDB are employed. In comparison with other studies and known techniques that use only indices or symptom scores to evaluate patients, it is believed that previously published data with these two questionnaires can provide more clinical relevant information in patient counseling.

However, it is generally agreed that questionnaire alone is not accurate sufficiently to discriminate patients with or without SDB but could be useful only in prioritizing patients for split-night PSG. The reported sensitivity of questionnaire varies from 72% to 96% in predicting OSAS, with specificity as low as 13% to 54%. The highest specificity of 0.77 reported from Berlin questionnaire was challenged because of its underestimation by using 4-channel sleep monitor as validation golden standard. In the first tier screening of the present invention, the strategy is to maximize the screening sensitivity. The AUC of the ROC curve reaches the level of 0.88, which is compatible with the reported data of 0.55 to 0.83 from similar studies in the literatures. With a calculated probability of 0.6, it is included as many patients (94.93%) as possible that probably have OSAS. Using the algorithm of the present invention, seventeen (17) patients will be exempted from PSG because their risks of having OSAS are so low; and one (out of 355) patient with true OSAS will be missed (Table 5).

Pulse oximetry is another frequently used tool for the screening of OSAS with great economical benefit. The Technology Assessment Task Force of the Society of Critical Care Medicine 1993 report indicated that pulse oximetry is a non-invasive tool to measure oxygen saturation with a high degree of accuracy over the range of 80% to 100% saturation. The 1995 British Thoracic Society report concluded that pulse oximetry criteria are highly specific when positive (specificity=100%), but may miss patients with hypopnic arousal without significant oxygen desaturation (sensitivity=31%). The Minota-Pulsox-3i that is used in the present invention, is designed specifically for the screening of OSAS to eliminate body movement artifact and to increase its prediction specificity. In the second tier screening, the strategy according to the present invention is to maximize the screening specificity. Even through the differences among DI2, DI3, and DI4 are small, it is found that the highest AUC of 0.951 indicates DI3 is the ideal threshold against RDI≧30. The desaturation index of 3% we use in this 2^{nd}-tier screening yield a sensitivity of 0.57 and a specificity of 0.96, which are comparable with what was reported by Golpe et al. (for RDI≧40.5 , specificity 97%). With a calculated probability of 0.5, 60% of patients that are not likely to have severe OSAS can be identified. Using the algorithm of the present invention, thirty-six (36) out of one hundred (100) patients will definitely need early PSG because their risks of having severe OSAS are high and four out of one hundred patients will be recruited for unnecessary sleep study (Table 7).

Since neither questionnaires nor pulse oximeter is ideal individually when used alone, some prior references have advocated the usefulness of pulse oximetry to establish the diagnosis of OSA and highlighted the value of clinical score to improve the sensitivity of screening tool. Schafer et al claimed that a combination of clinical features, questionnaires and pulse oximetry may achieve a model specificity of 92%. Rauscher et al used clinical predictors and oximeter to establish a OSAS screening model with sensitivity of 94%, specificity of 45% to predict an apnea-hypopnoea index above 10, sensitivity of 95% and specificity of 41% to predict an apnea-hypopnoea index above 20. In this study we seek to optimize the prediction algorithms by developing a stepwise, two-tier screening model. By using ESS and SOS, 4.8% (18 out of 355, including 1 false negative) of patients are exclude from PSG testing at the first tier screening since their risks of having OSAS is low. By using pulse oximeter, 40% (40 out of 100, including 4 false alarm) of patients are prioritized for early PSG testing since their risks of having severe OSAS is high. These cost-effective data are equivalent to what have been reported by Keenan et al. and by Gurubhagavatula et al. Keenan et al. Confidently diagnosed OSA in 20% and exclude OSA in 5% of patients based on their prediction model using questionnaire, physical examination and home oximetry. Gurubhagavatula et al's 2-stage model altogether excluded 8% of patients from sleep studies, but prioritized up to 23% of subjects to receive in-laboratory studies with 95% sensitivity for OSAS and 97% specificity for severe OSAS.

In conclusion, the two tier screening model of the present invention can jointly exclude 4.8% of innocent subjects from sleep studies, but can prioritize up to 40% of severe OSAS patients to receive complete in-laboratory PSG with 0.603 sensitivity for OSAS and 0.966 specificity for severe OSAS. It is believed that the screening efficiency and utility can be further improved when applied to general population, given the referred nature of SDB patients used in this validation study. The prediction algorithm of the present inventive model is sufficiently accurate that is feasible for large-scale community or occupational SDB screening in the future.

Although the present invention has been described with reference to what is believed to be the best mode for carrying out the present invention, it is apparent to those skilled in the art that a variety of modifications and changes may be made without departing from the scope of the present invention which is intended to be defined by the appended claims

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A two tier method for screening a predetermined number of patients with sleep-disordered breathing, comprising:
(A) collecting personal data for each patient;
(B) doing at least one form of survey for each patient;
(C) employing multiple regression to obtain a first-tier estimated sleep respiratory disturbance index (RDI) with the clinical data and survey forms data collected previously;
(D) comparing the first-tier estmated RDI with a threshold to exclude first group of patients with a second group of patients remains for further examination;
(E) using a pulse oximeter to measure desaturation of oxygen for each patient of the remaining second group of patients to obtain sleep oxygen desaturation events;
(F) obtaining a second-tier estimated RDI based on the sleep oxygen desaturation events; and
(G) comprising the second-tier estimated RDI with a second threshold to determine patient that are truly of sleep-disordered breathing.

2. The method as claimed in claim 1, wherein the personal data collected in step (A) includes gender, age, and body mass index (BMI).

3. The method as claimed in claim 1, wherein the form survey performed in step (B) comprises Snore Outcomes Survey (SOS).

4. The method as claimed in claim 3, wherein the SOS includes eight items for evaluating duration, severity, frequency, and consequence of problems associated with sleep-disordered breathing on a Likert scale, and each item having five to six response and wherein the SOS score is transformed into a scale ranging from 0 to 100.

5. The method as claimed in claim 1, wherein the form survey performed in step (B) comprises Epiworth Sleepiness Scale (ESS).

6. The method as claimed in claim 5, wherein the ESS includes eight items used to evaluate average sleep propensity, each item having a score ranging from 0 to 3 and total score ranging 0 to 24.

7. The method as claimed in claim 1, wherein the first-tier threshold is five and wherein the patient having a first-tier estimated RDI greater than the first-tier threshold is considered a patient of sleep-disordered breathing.

8. The method as claimed in claim 1, wherein receiver operating characteristic curve is employed to determine diagnostic threshold for SOS and ESS, wherein area under curve is demonstrated; sensitivity, specificity, positive and negative predictive values of different possible SOS and ESS combinations is calculated; and boot-trap technique is employed to identify a cut-off point.

9. The method as claimed in claim 1, wherein desaturation of oxygen by 2, 3, and 4%, namely oxygen desaturation index of 2, 3, and 4%, are defined as an episode of respiratory disturbance.

10. The method as claimed in claim 7, wherein a multiple and logistic regression is used to determine the second RDI.

11. The method as claimed in claim 10, wherein the second-tier estimated RDI greater than the second-tier threshold is considered a patient of server sleep-disordered breathing.
